(19) United States
(12) Patent Application Publication
(10) Pub. No.: US 2022/0117716 A1
(43) Pub. Date: Apr. 21, 2022

(54) INTRAVESICULAR DEVICE FOR CONTROLLING URINARY INCONTINENCE

(71) Applicant: Innoventions LTD., Or Akiva (IL)

(72) Inventor: Daniel YACHIA, Herzliya Pituach (IL)

(73) Assignee: Innoventions LTD., Or Akiva (IL)

(21) Appl. No.: 17/417,433

(22) PCT Filed: Dec. 24, 2019
(86) PCT No.: PCT/IL2019/051403

§ 371 Date: Jun. 23, 2021

Related U.S. Application Data
(60) Provisional application No. 62/784,559, filed on Dec. 24, 2018.

(51) Int. Cl. A61F 2/00; A61B 1/00; A61B 1/307; A61B 5/20

(52) U.S. Cl. CPC A61F 2/0027; A61B 1/00082; A61B 1/307; A61B 1/00016; A61F 2250/0067; A61F 2210/009; A61F 2230/0071; A61F 2250/0002; A61B 5/202

(57) ABSTRACT

The invention provides a device for treating urinary incontinence having a fillable expandable balloon made of a flexible material. The wall of the balloon has at least one recess and a valve. A central core located in the lumen of the balloon encloses a central channel having one or more openings and one or more magnetable elements there being.

### BACKGROUND OF THE INVENTION

The following prior art publications are considered to be relevant for an understanding of the background of the invention:

US Pat. Nos. 6,293,923, 4,258,705, 4,834,704, 4,850,963, 4,871,542, 4,925,446, 5,019,032, 5,030,199, 5,188,109, 5,234,409, 5,443,470, 5,513,659, 5,579,781, 5,604,531, 5,704,353, 5,749,845, 5,806,527, 5,984,860, 6,039,967, and 6,139,535.

European Patent Publication No. 0667115.

### FIELD OF THE INVENTION

The invention is in the field of medical devices. More specifically, the invention relates to devices for the treatment of urinary bladder disorders.

Urinary incontinence has serious economic, health, social and psychological consequences. Fear of incontinence and odors in public cause incontinent people to severely restrict their social activities. The impact on the mental health of the affected people may be even more devastating than the social and health consequences. They suffer severe embarrassment, loss of self-esteem, depression and anxiety.

Urinary incontinence has been treated surgically and pharmacologically. Various conservative/behavioral treatments are also known such as pelvic floor muscle exercises, bladder training, biofeedback, vaginal cones, low-frequency electrostimulation of pelvic floor muscles, intravaginal bladder neck support pessaries, urethral meatus suction cups and intraurethral devices.

Devices which have been used to obtain almost immediate dryness in incontinent people include urethral plugs and valve catheters.

U.S. Pat. No. 6,293,923 describes a system comprising an expandable balloon to be inserted into the urinary bladder that may be used for the intermittent sealing of the urinary bladder outlet and the prevention of involuntary urine leakage. Sealing the urinary bladder outlet involves lodging the balloon in the outlet so as to seal it. Unsealing the outlet to allow voiding of the bladder involves dislodging the balloon from the outlet.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a device comprising a balloon for treating urinary incontinence in accordance with one embodiment of the invention;

FIG. 2 shows the balloon of the device of FIG. 1;

FIG. 3 shows filling of the device of FIG. 1 with a fluid;

FIG. 4 shows the device of FIG. 1 in a sealing position in the urinary bladder of a female individual; and

FIG. 5 shows a close-up of the device of FIG. 1 near the bladder opening.

### DESCRIPTION OF THE INVENTION

The present invention provides a fillable balloon for insertion into the urinary bladder of an individual, for the treatment of urinary incontinence.

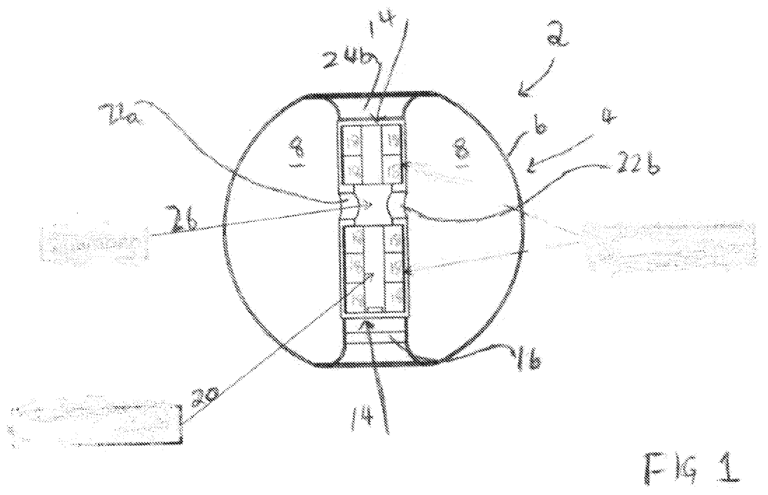

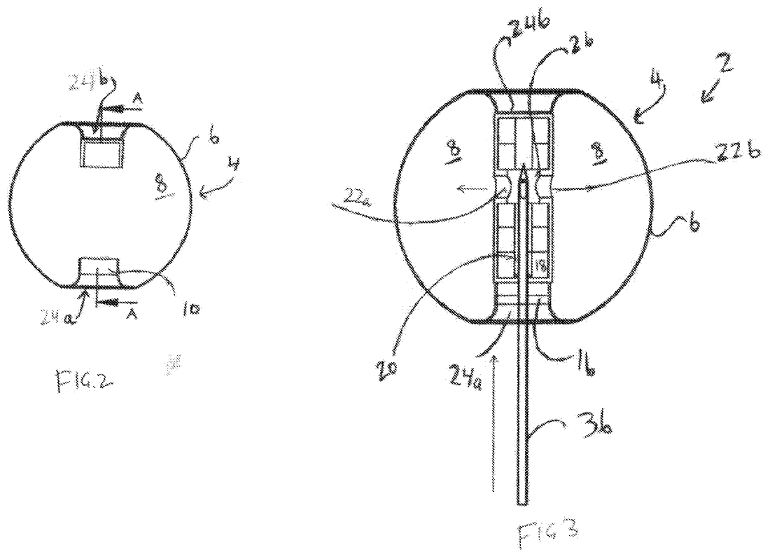

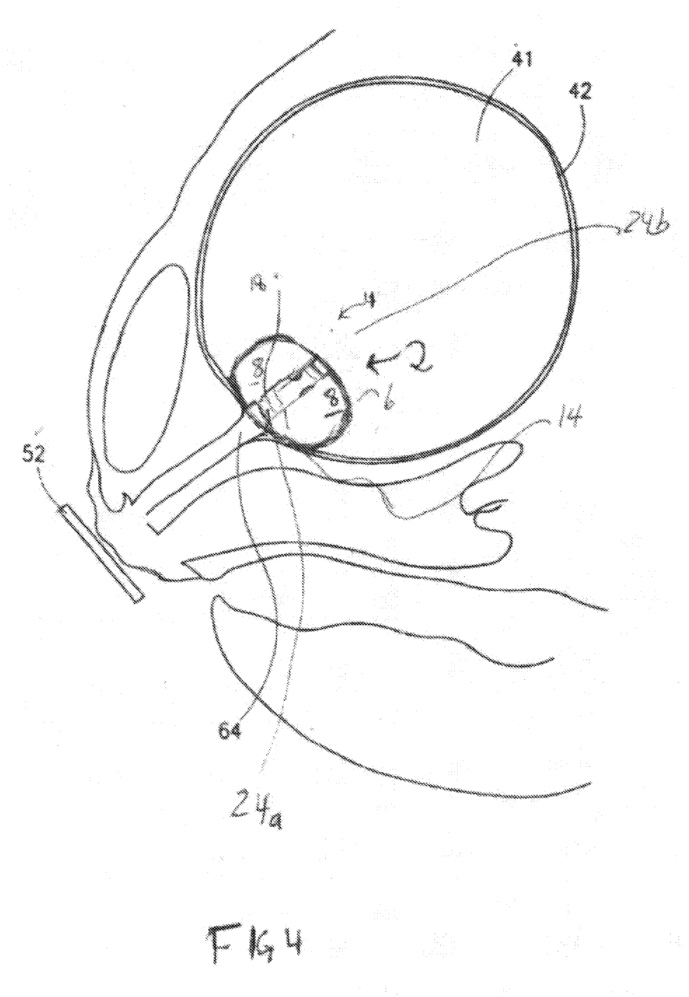

Reference is now made to FIG. 1 which shows a device 2 for treating s urinary incontinence in accordance with one embodiment of the present invention. The device 2 comprises a fillable balloon 4 shown in cross section in FIG. 1. The fillable hollow balloon is shown isolated in FIG. 2, and has a wall 6 made of a flexible biocompatible material enclosing a lumen 8. The wall of the balloon 2 has two recesses 24a and 24b that are diametrically opposed from each other.

The wall 6 of the balloon 4 can be made from any biocompatible, flexible material such as silicone. The wall of the balloon 4 has an opening that will eventually be sealed closed. Prior to sealing the balloon, a central core 14, shown in FIG. 1, is inserted into the lumen 8 of the balloon 4. A self-sealing is valve 16 is inserted into the opening of the wall of the balloon which is used to fill the balloon, as explained below. The valve 10 may, be for example a duck-bill type valve or a ball valve.

The core 14 is made from a flexible material and comprises one or more magnetable elements 18. In the embodiment of FIG. 1, the magnetable elements 18 are toroidal rings that are stacked along the longitudinal axis of the central core 14. The magnetable elements 18 surround a central channel 20. The inventors have found that using two or more independent stacked toroidal magnetable elements confers flexibility to the central core 14, which can be of advantage when the device 2 is inserted through the male urethra to the urinary bladder, as explained below. A section of the central core 14 not surrounded by any magnetable elements 18 is referred to herein as the “chamber” 26. Two openings 22a and 22b in the wall of the chamber 26 allow a fluid introduced into the central channel to leave the central core 14 and enter the lumen 8 of the balloon 4, as explained below.

The device 2 is delivered through the urethra into a urinary bladder with the lumen 8 of the balloon 4 empty, and the wall 6 of the balloon 4 collapsed onto the central core 14. Delivery of the device 2 is done using a delivery applicator, for example, as disclosed in U.S. Pat. No. 6,293,923. The magnetable elements within the core 18 confer longitudinal incompressibility to the central core, thus providing better pushability of the device through the urethra. Inside the bladder, the device 4 is filled with a fluid to its original volume or less and then released from the delivery catheter.

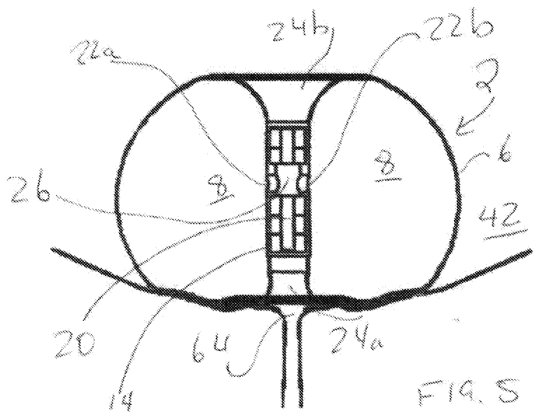

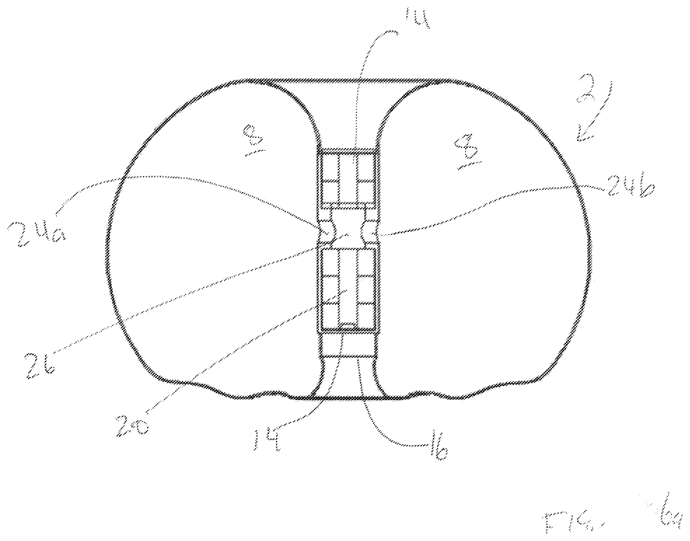

As shown in FIG. 3, for filling the balloon 4, the cannula 36 of a syringe is delivered through the urethra to the urinary bladder, and inserted through the valve 16 into the central channel 20 of the central core. A fluid is injected from the syringe through the cannula 36 into the central core 20 that leaves the central core 20 through the openings 22a and 22b of the chamber 26, and fills the lumen 8 of the balloon 4. This causes the balloon 4 to be filled to its original volume or less. After filling, the cannula 36 is withdrawn, and the valve 16 seals itself. The fluid can be any biocompatible fluid, which may be presterilized such as air, water, saline or an oil such as liquid paraffin. The filled balloon may have a specific gravity that is less than that of urine or it may have a specific gravity that is greater than that of urine.

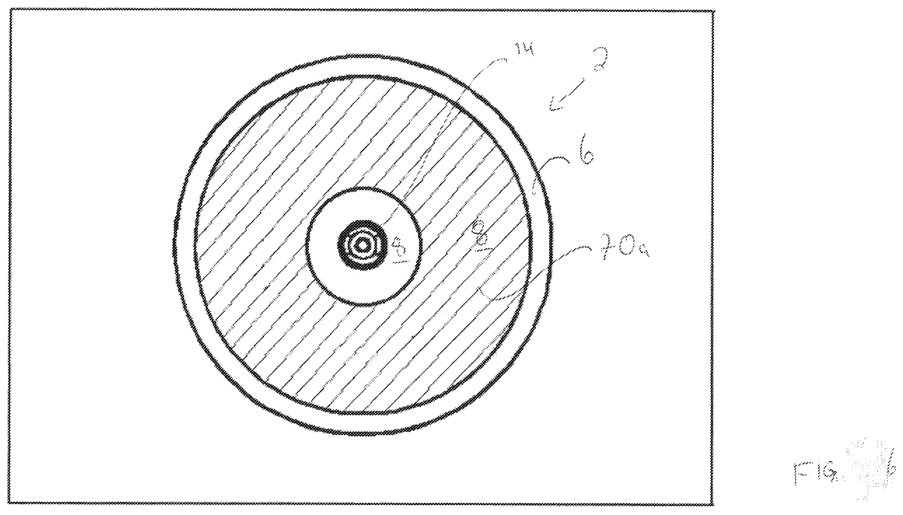

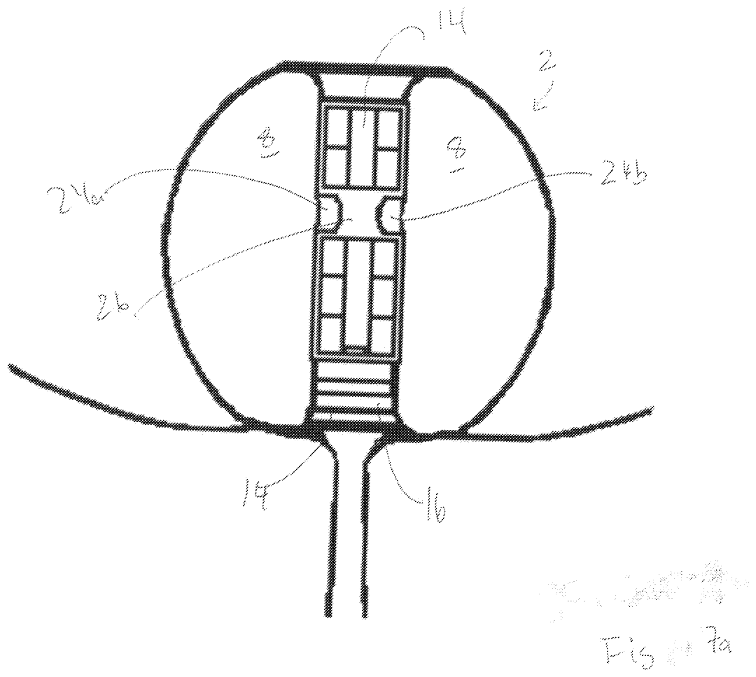

FIG. 4 shows the device 2 after deployment in the urinary bladder 42 of a female individual. An external magnet 52 is used to positon the device 2 in the bladder outlet 64. FIG. 5 shows a close-up of the device 2 positioned in the urinary bladder outlet 64. In this position, the device 2 is in a sealing position, and leakage of urine is prevented. The inventors have found that recessing the valve 16 from the surface of the wall 6 of the filled balloon tends to be more comfortable for the individual when the device 2 is in the sealing position shown in FIG. 4. The inventors have further found that when the balloon 4 is filled, the balloon 4 should preferably not be filled to its maximum capacity. When the balloon 4 is not filled to its maximum capacity, the wall 6 of the balloon 4 is not tautand the balloon 4 is flaccid, rendering the balloon supple and better suited to conform to the irregular surface of the bladder opening 64 and the nearby bladder surfaces around the bladder outlet. Under these conditions, the surface area of contact between the device 2 and the bladder surface (the “footprint” of the device 2 on the bladder surface“) is enhanced, and the balloon provides a better seal.

When it is desired to void the bladder of urine, the external magnet 52 is removed. If, after filling of the lumen 8 the device 2 has a specific gravity less than that of urine, the device 2 will spontaneously float in the urine thus opening the bladder opening 64 and allowing voiding. If, after filling of the lumen 8 the device 2 has a specific gravity greater than that of urine, the device 2 can be moved to a non-sealing position in the bladder by placing the external magnet 52 on the individual's abdomen.

When it is desired to remove the device 2 from the body, the cannula which is within a magnetic tipped retrieval device to engage the magnetic element of the balloon 36 and connected to an empty syringe or a vacuum chamber is delivered through the urethra to the urinary bladder and the balloon is engaged. The cannula 16 is inserted into the central channel 20 of the central core (refer again to FIG. 3). The fluid in the lumen and central core of the device is withdrawn into the syringe or the vacuum chamber. In this way, the fluid is not released into the urinary bladder, which could irritate the wall of the bladder. Having the evacuation cannula within the channel surrounded by the central core also prevents accidental injury of the bladder by the cannula. The empty device 2 can then be removed from the body using the magnetic retrieval device.

The external magnet 52 can be incorporated into a hygienic pad or in the underwear of the individual.

The balloon 4 may be adapted to store one or more substances and releasing them into the urinary bladder. The one or more substances may be stored in the lumen 8 of the balloon, or in the wall 6 of the balloon. The balloon may further comprise imaging means for imaging the urinary bladder and a transmitter for transmitting signals from the imaging means to a receiver. The balloon may also include one or more monitoring means for monitoring urinary bladder parameters and contents of the bladder. The parameters may be, for example, bladder pressure; urine temperature; urine density; and urine composition. The balloon may further comprise a transmitter for transmitting signals from the monitoring means to a receiver. The balloon may also include one or more electrodes.

1. A device for treating urinary incontinence comprising:
(a) a fillable expandable balloon having a wall made of a flexible material enclosing a lumen, the wall of the balloon having at least one recess, there being a valve in the wall of the balloon; and,
(b) a central core located in the lumen of the balloon having an end adjacent to the valve, the central core comprising a wall enclosing a central channel and one or more magnetable elements, there being one or more openings in the wall of the central channel.

2. The device according to claim 1, comprising two or more magnetable elements.

3. The device according to claim 3, wherein the magnetable elements are toroidal rings.

4. The device according to claim 1, wherein the central core surrounding the valve and the magnetic elements is made from a flexible material.

5. The device according to claim 1, wherein the central core is longitudinally incompressible.

6. The device according to claim 1, wherein the balloon is filled with a fluid.

7. The device according to claim 6, wherein the wall of the filled balloon is not taut.

8. The device according to claim 7, wherein the filled balloon is flaccid.

9. The device according to claim 1, wherein the balloon is adapted to store one or more substances and releasing the substances into the urinary bladder.

10. The device according to claim 1, wherein the balloon further comprises imaging means for imaging the urinary bladder.

11. The device according to claim 1, wherein the balloon further includes one or more monitoring means for monitoring urinary bladder parameters or measuring contents of the bladder.

12. The device according to claim 11, wherein the balloon further comprises a transmitter for transmitting signals from the monitoring means to a receiver.

13. The device according to claim 10, wherein the balloon further comprises a data collector for collecting data generated by imaging means or monitoring means associated with the balloon.

14. The device according to claim 1, wherein the balloon further comprises one or more surface or antenna shaped electrodes.

15. A system for treating urinary incontinence comprising:
(a) a device for treating urinary incontinence comprising:
a fillable balloon having a wall made of a flexible material enclosing a lumen, the wall of the balloon having at least one recess, there being a valve in the wall of the balloon; and,
a central core located in the lumen of the balloon having an end adjacent to the valve, the central core comprising a wall enclosing a central channel and one or more magnetable elements, there being one or more openings in the wall of the central channel.
(b) a magnet configured to be applied to a body surface of an individual.

16. The system according to claim 15, further comprising a delivery catheter configured to deliver the device to a urinary bladder of an individual.

17. The system according to claim 15, further comprising a retrieval device configured to remove the device from a urinary bladder of an individual.

18. The system according to claim 15, further comprising a syringe configured to introduce a fluid into the balloon through one of the valves and to remove fluid from the balloon through one of the valves.

19. A method for treating urinary incontinence comprising:
(a) inserting into a urinary bladder of an individual a device for treating urinary incontinence comprising:
an expandable balloon having a wall made of a flexible material enclosing a lumen, the wall of the balloon having at least one recess, there being a valve in the wall of the balloon; and,
a central core located in the lumen of the balloon having an end adjacent to the valve, the central core comprising a wall enclosing a central channel and one or more magnetable elements, there being one or more openings in the wall of the central channel.
(b) filling the lumen of the balloon with a fluid; and
(c) positioning the device in an opening of the urinary bladder using a magnet located outside of the bladder;
(d) removing the device from the opening of the urinary bladder when the bladder is to be voided.

20. The method according to claim 19, wherein the fluid is selected from a biocompatible fluid, air, water, saline or an oil.

21. The device according to claim 19, wherein the inflated balloon has a specific gravity that is less than the specific gravity of urine.

22. The method according to claim 19, wherein the inflated balloon has a specific gravity that is greater than or equal to the specific gravity of urine.

23. The method according to claim 19, wherein the wall of the balloon is not taut when the balloon is filled.

24. The method according to claim 23, wherein the balloon is flaccid when filled.
